(19) United States
(12) Patent Application Publication
(10) Pub. No.: US 2023/0074431 A1
(43) Pub. Date: Mar. 9, 2023

(54) DEVICES AND METHODS FOR DETERMINING PHOSPHATE LEVELS IN NATURAL WATER

(71) Applicant: MASSACHUSETTS INSTITUTE OF TECHNOLOGY, Watertown, MA (US)

(72) Inventors: Geehoon PARK, Watertown, MA (US); Ian HUNTER, Lincoln, MA (US)

(21) Appl. No.: 17/799,261

(22) PCT Filed: Feb. 11, 2021
(86) PCT No.: PCT/US2021/017733

§ 371 Date: Aug. 11, 2022

Related U.S. Application Data
(60) Provisional application No. 62/972,994, filed on Feb. 11, 2020.

(51) Int. Cl. G01N 27/404; G01N 33/18; G01N 27/30

(52) U.S. Cl. CPC G01N 27/404; G01N 33/18; G01N 27/30

(57) ABSTRACT

Devices and methods for measuring phosphate levels in a fluid, such as natural water, are disclosed. The devices and methods rely upon the anodic dissolution of molybdenum to generate a reagent from a phosphate-containing fluid, which is then measured electrochemically to determine a level of phosphate ions in the fluid. The different embodiments of devices used for performing this technique are transportable and have extended lifetimes when compared to existing devices used to measure phosphate levels in fluid. They can be used in situ at the source of a site to generate results within about two minutes, within about thirty seconds, and even within about ten seconds. They also consume much less energy and molybdenum per measurement. The disclosed embodiments include devices having one or two molybdenum electrodes, with one of the electrodes disposed near a working electrode. Various methods for determining phosphate levels in fluids are also provided.

### CROSS REFERENCE TO RELATED APPLICATIONS

The present application claims priority to and the benefit of U.S. Provisional Patent Application No. 62/972,994, filed Feb. 11, 2020, and titled “Devices and Methods for Determining Phosphate Levels in Natural Water,” and which is incorporated by reference herein in its entirety.

### FIELD

The present disclosure relates to devices and methods for detecting phosphate levels in a fluid (e.g., natural water), and more particularly relates to the use of one or more molybdenum (Mo) electrodes to allow for in situ reagent generation to measure such phosphate levels in a small, transportable device.

### BACKGROUND

Phosphorus is an essential nutrient element used by all living organisms for their growth and energy transport. Humans and all other vertebrates need phosphorus to build their bones and teeth which contain up to 50% by volume and 70% by weight of hydroxyapatite, Ca₁₀(PO₄)₆(OH)₂. Additionally, many other phosphorus compounds play important roles in fundamental biochemical functions. For instance, nucleotides act as precursors in DNA and RNA, adenosine triphosphate (ATP) can be an important source of energy to many biological systems, phospholipid is often considered the main characteristic component of a cell wall membrane, and the buffering capability of phosphate ions (PO₄³⁻) can be important in maintaining the pH of intracellular environment.

Despite its importance to many living organisms, excess level of phosphorus in aquatic environment can be detrimental, since phosphorus, along with nitrogen and carbon, is understood to be the main nutrient that causes eutrophication in water. Eutrophication is one of the six major environmental problems in lakes, estuaries and coastal waters throughout the world. It describes the phenomenon in water where the rate of supply of organic matter to an ecosystem is increased. The over-supply of the organic matter to the water can stimulate the growth of different types of micro-organisms including dense nuisance and toxic algae causing decrease of the light penetration and beneficial submerged aquatic vegetation. Further, the growth of micro-organisms can reach the state where it cannot be balanced by the consumption from species in the higher rank of the food web, and that excess organic matter sinks to the bottom of water. As a result, the oxygen level in the water decreases as it is decomposed by bacteria which consumes oxygen in the process. The oxygen can be critical to every living organism in water and the decrease of it (hypoxia, <3 milligrams/Liter of O₂ in water) or the absence of it (anoxia) can significantly disturb the ecosystem in water by causing the death of fish and other important organisms, the degradation of their habitat, and the alteration of their migration pattern. Moreover, from an economic perspective, it can lead to the decrease in fishery production and negatively impact the life pattern and tourism of local communities, among other problems.

The importance of the monitoring of phosphorus level in natural waters mainly comes from the effect of phosphorus control on the management of the eutrophication. In water, phosphorus never exists by itself. It forms either inorganic orthophosphate (phosphate), condensed polyphosphate, or organic phosphate. Even with its variously defined forms, the total phosphorus level in natural water is typically determined by decomposing all the different phosphorus-related compounds into inorganic phosphate and measuring its concentration. Numerous studies have developed technologies to detect phosphate or other types of phosphorus-related compounds. The detection mechanisms of these technologies include spectrophotometry, electrochemistry, fluorescence spectroscopy, infrared/Raman spectroscopy, NMR spectroscopy, and enzyme-based biology. Although the detection range of each technology and the type of water to which the technologies can be applied cover a wide range, one method for determining phosphorus level in natural water is a spectrophotometric determination of phospho-molybdenum blue (PMB) molecule (PMB method). In this method, phosphate ions and acidified molybdate ions form 12-molybdophosphoric acid (12-MPA) in an acidic environment. 12-MPA is further reduced by the reductant into the PMB molecule of which intensity is correlated with the concentration of phosphate ions.

Despite the fact that the PMB method can be applied to most natural waters with a good sensitivity and selectivity to phosphate ions, there are still great demands for the improvement of the technology. For example, the demands for a reliable in situ and portable measurement device has dramatically increased as obtaining the temporal and spatial information of the phosphorus level can be important in water quality management. However, the current technology typically requires manual sampling, and the transport and storage of the sample, followed by laboratory-based analysis. To the extent any portable phosphate measurement devices exist, these devices suffer from several problems, including that the footprint of the device increases with the increase of its working time. This is simply because an increase in the number of measurements yields an increase in the volume of reagents for the PMB method.

While attempts have been made to overcome the limitations of existing phosphate level detection devices, such devices have included complications such as complex mechanical parts and/or electrical systems that do not allow for such devices and systems to be used in an easy, portable manner. For example, some existing devices that utilize molybdenum oxidation for in situ reagent generation include devices having chambers with volumes of 365.4 μL and 91 μL. Such devices can require significant energy (e.g., 18 Joules of energy per measurement), and time (e.g., 180 seconds) to induce the level of oxidation necessary for measurements to be made. In other words, most of the energy is consumed in generating a considerable amount of protons through the oxidation to lower the pH of the reaction environment for the formation of 12-MPA. A reaction environment with high pH can easily lead acidified molybdate ions to form 12-molybdosiliacid (12-MSA), instead of 12-MPA, under the presence of silicate ion, a major interference ion in the PMB method. Because 12-MPA and 12-MSA exhibit similar electrochemical behaviors, preventing such interference by providing excess protons, thereby increasing the energy consumption, becomes inevitable in designing these devices.

Additionally, the time to actually make a measurement can be on the order of about 80 minutes, at least because the slow diffusion of acidified molybdate ions and 12-MPA makes it difficult to have a homogeneous reaction environment and detection environment for 12-MPA. To the extent devices have been developed capable of reducing the 80-minute wait time, they often utilize additional mechanical and/or electrical components (e.g., a pump) to mix or homogenize the resulting solution to make the analysis, and that still can take at least 5 minutes. This increase in complexity to improve wait times is not an ideal trade-off, as in addition to extra components, it causes the volume of the device to increase and raises the energy consumption of the device. Limiting factors of existing devices that may be considered to be portable include the number of measurements a device can make without requiring a reagent refill, and, relatedly, the volume of reagents that can be loaded in the device.

Accordingly, there is a need for devices and methods that allow for phosphate levels of a fluid (e.g., natural water) to be measured in situ using transportable, easy to use devices that make quick measurements, are energy-efficient, and minimize an amount of reagent used on a per-measurement basis so that the shelf-life of the device without requiring a refill is extended beyond current capabilities.

### SUMMARY

The present disclosure provides for phosphate level measuring devices that have a minimal footprint with respect to size, energy consumption, time to make measurements and phosphate level determinations, and the amount of reagent needed per measurement, meaning more measurement can be made with the same device. The devices provided for herein utilize in situ reagent generation by anodic dissolution of molybdenum and detecting 12-molybdophosphoric acid (12-MPA) to make determinations about phosphate levels of the fluid being tested. The devices and methods provided for herein are useful with respect to natural water, and are particularly useful with respect to fresh water. Phosphate levels in other waters can also be measured, including but not limited to surface water from bodies such as ponds or lakes, seawater, and drinking water. Still further, the teachings of the present disclosure can be adapted for use with measuring phosphate levels in other fluids, typically liquids.

The devices, and related methods, allow for in situ generation and/or supply of the reagents for making phosphate level determinations (e.g., molybdate and protons) by the anodic dissolution of molybdenum metal. At least three different designs of phosphate level measuring devices are provided for in the present disclosure. One illustrated device (see, e.g., FIG. 3) utilizes a two molybdenum electrode configuration that reduces energy and material consumption and allows for faster detection. A second illustrated device (see, e.g., FIG. 4) utilizes a single molybdenum electrode configuration to even further reduce energy and material consumption while also allowing for even faster detection that the first illustrated device. A third illustrated device (see, e.g., FIG. 5) utilizes an open-cell single molybdenum configuration that reduces the complexity of the system while allowing for reduced energy and material consumption and even faster detection that the second illustrated device. Additional details about these embodiments, and the configurations of phosphate level measuring devices, is discussed in greater detail below.

The method of using the devices involves supplying the fluid, which includes phosphate ions, to a device that includes a molybdenum electrode. The molybdenum electrode is oxidized to supply molybdate ions to the fluid, the ions having a low pH to form 12-molybdophosphoric acid (12-MPA). A redox response results and is measured using an electrochemical measurement that includes one or more of a working electrode(s), a reference electrode(s), and a counter electrode(s). In at least some of the provided embodiments, the working electrode is disposed near a molybdenum electrode, typically near the second molybdenum electrode in embodiments that use at least two molybdenum electrodes, the second molybdenum electrode being disposed further downstream from a reaction that occurs by way of oxidation of a first molybdenum electrode (as shown in at least some of the embodiments, distal of the first molybdenum electrode).

One exemplary embodiment of a phosphate level detection device includes a first chamber, a first molybdenum electrode, and a working electrode. The first molybdenum electrode is at least partially disposed with the first chamber, and the working electrode is also at least partially disposed within the first chamber. Further, the working electrode is positioned within about 100 micrometers of the first molybdenum electrode. The device is configured such that oxidation of the first molybdenum electrode in the presence of a fluid that includes phosphate ions results in the formation of a 12-molybdophosphoric acid. Notably, the first chamber can have an enclosed configuration or an open configuration, depending on the desired set-up.

In some embodiments, the device can include a second chamber, a second molybdenum electrode, and a proton exchange membrane. In at least some such embodiments, each of the first and second chambers can be enclosed chambers, the second molybdenum electrode can be at least partially disposed in the second chamber, and the proton exchange membrane can be disposed between the first and second chambers. Further, the device can be configured such that oxidation of the second molybdenum electrode in the presence of a fluid results in protons migrating from the second chamber, across the proton exchange membrane, and to the first chamber to reduce a pH level of fluid disposed in the first chamber. More specifically, in some embodiments, the device can be configured such that the protons that migrate from the second chamber, across the proton exchange membrane, and to the first chamber reduce the pH level to a range of about 0.8 to about 1.2, such as to about 1. In embodiments that include a proton exchange membrane disposed between first and second chambers, the first molybdenum electrode and the working electrode can be disposed directly adjacent to the proton exchange membrane.

The phosphate level detection device can also include a reference electrode and/or a counter electrode. The reference and/or counter electrodes, in conjunction with the working electrode, can be configured to make electrochemical determinations of a phosphate level of a fluid disposed in the first chamber when the first molybdenum electrode is oxidized. In at least some embodiments that include both a reference electrode and a counter electrode, the first chamber can be an open chamber and each of the first molybdenum electrode, the working electrode, the reference electrode, and the counter electrode can be at least partially disposed within bounds defined by walls of the first chamber. In such instances, the device can be configured to operate in an open-cell configuration.

In some embodiments the device can have a total chamber volume of about 6 microliters or less. This volume can be about 1.5 microliters or less in some instances. In some embodiments a molybdenum consumption level can be approximately 0.08 milligrams or less per measurement. This consumption level can be approximately 0.0008 milligrams or less per measurement in some instances. In some embodiments an energy consumption level for oxidation of the first molybdenum electrode can be approximately 0.2 Joules or less per measurement for each 1 millimeter² of exposed first molybdenum electrode surface area. This consumption level can be approximately 0.00025 Joules or less per measurement for each 1 millimeter² of exposed first molybdenum electrode surface area in some instances. In some embodiments a phosphate level detection time of the device can be approximately two minutes or less in the absence of stirring the 12-molybdophosphoric acid. This time can be approximately 30 seconds or less in the absence of stirring the 12-molydophosphoric acid in some instances, or even approximately 10 seconds or less in the absence of stirring the 12-molydophosphoric acid in some instances.

One exemplary method for determining phosphate levels in a fluid includes oxidizing a first molybdenum electrode that is at least partially disposed in a first chamber of a phosphate level detection device to form a 12-molybdophosphoric acid from a fluid having phosphate ions disposed in the fluid, and determining a level of phosphate ions present in the fluid from which the 12-molybdophosphoric acid is formed. The determination is done using an electrochemical set-up, and the electrochemical set-up includes a working electrode disposed within about 100 micrometers of the first molybdenum electrode. Notably, the first chamber can have an enclosed configuration or an open configuration, depending on the desired set-up.

The method can further include causing the fluid having phosphate ions disposed therein to enter the first chamber. The fluid can come from a fluid source that is at a location at which the oxidizing and determining actions are performed such that determining a level of phosphate ions present in the fluid from which the 12-molybdophosphoric acid is formed occurs in situ.

In some embodiments, the method can further include oxidizing a second molybdenum electrode that is at least partially disposed in a second chamber to cause protons to migrate from the second chamber, to the first chamber, to reduce a pH level of the fluid disposed in the first chamber. In such embodiments, the first and second chambers can be enclosed. In at least some such embodiments, the method can further include causing the fluid having phosphate ions disposed therein to enter the second chamber. The fluid can come from a fluid source that is at a location at which the oxidizing and determining actions are performed such that determining a level of phosphate ions present in the fluid from which the 12-molybdophosphoric acid is formed occurs in situ.

The migration of the protons from the second chamber to the first chamber can reduce the pH level of the fluid disposed in the first chamber to a range of about 0.8 to about 1.2, for example to about 1. In at least some embodiments, the first molybdenum electrode and the working electrode can be disposed directly adjacent to the second chamber.

In some embodiments a total chamber volume of the phosphate level detection device can be about 6 microliters or less. This volume can be about 1.5 microliters or less in some instances. In some embodiments approximately 0.08 milligrams of molybdenum or less is consumed per measurement. This consumption level can be approximately 0.0008 milligrams or less per measurement in some instances. In some embodiments approximately 0.2 Joules or less of energy for each 1 millimeter² of exposed first molybdenum electrode surface area can be consumed per measurement. This consumption level can be approximately 0.00025 Joules or less of energy for each 1 millimeter² of exposed first molybdenum electrode surface area per measurement in some instances. In some embodiments determining a level of phosphate ions present in the fluid from which the 12-molybdophosphoric acid is formed using an electrochemical set-up consumes approximately two minutes or less of time in the absence of stirring the 12-molybdophosphoric acid. This time can be approximately 30 seconds or less in the absence of stirring the 12-molydophosphoric acid in some instances, or even approximately 10 seconds or less in the absence of stirring the 12-molydophosphoric acid in some instances.

Another exemplary method for determining phosphate levels in a fluid includes sampling a fluid having phosphate ions disposed in the fluid from a fluid source. The method further includes generating a reagent from the fluid having phosphate ions disposed in it due to anodic dissolution of molybdenum metal and determining a level of phosphate ions in the fluid from which the reagent is generated. The actions of sampling, generating, and determining are all performed at the location of the fluid source. Thus, determining a level of phosphate ions in the fluid from which the reagent is generated occurs in situ. Notably, the method can be performed using set-ups with closed or open configurations, depending on the desired set-up.

The action of generating a reagent from the fluid having phosphate ions disposed in it due to anodic dissolution of molybdenum metal can include oxidizing a first molybdenum electrode that includes the molybdenum metal that is dissolved. In some such embodiments, a working electrode used in conjunction with determining a level of phosphate ions in the fluid from which the reagent is generated can be disposed within about 100 micrometers of the first molybdenum electrode.

The first molybdenum electrode can be disposed in a first enclosed chamber of a phosphate level determination device. In some such embodiments, the method can further include oxidizing a second molybdenum electrode disposed in a second enclosed chamber of the phosphate level determination device. A proton exchange member can be disposed between the first enclosed chamber and the second enclosed chamber, with the proton exchange member being able to cause protons to migrate from the second enclosed chamber to the first enclosed chamber to reduce a pH level of the fluid disposed in the first enclosed chamber. The migration of the protons from the second enclosed chamber to the first enclosed chamber can reduce the pH level of the fluid disposed in the first chamber to a range of about 0.8 to about 1.2, for example to about 1. In at least some embodiments, the first molybdenum electrode and the working electrode can be disposed directly adjacent to the second chamber.

In some embodiments a total chamber volume of the phosphate level detection device can be about 6 microliters or less. This volume can be about 1.5 microliters or less in some instances, for example when the first molybdenum electrode is disposed in a first chamber of a phosphate level detection device. In some embodiments approximately 0.08 milligrams of molybdenum or less is consumed per measurement. This consumption level can be approximately 0.0008 milligrams or less per measurement in some instances. In some embodiments approximately 0.2 Joules or less of energy for each 1 millimeter² of exposed first molybdenum electrode surface area can be consumed per measurement. This consumption level can be approximately 0.00025 Joules or less of energy for each 1 millimeter² of exposed first molybdenum electrode surface area per measurement in some instances. In some embodiments determining a level of phosphate ions present in the fluid from which the reagent is generated consumes approximately two minutes or less of time in the absence of stirring the 12-molybdophosphoric acid. This time can be approximately 30 seconds or less in the absence of stirring the fluid during the generating and determining actions in some instances, or even approximately 10 seconds or less in the absence of stirring the fluid during the generating and determining actions in some instances.

### BRIEF DESCRIPTION OF DRAWINGS

This disclosure will be more fully understood from the following detailed description taken in conjunction with the accompanying drawings, in which:

FIG. 1 is a schematic side view of one exemplary embodiment of a phosphate level measuring device having two molybdenum electrodes;

FIGS. 2A-2E illustrate one exemplary method of using the device of FIG. 1 to measure phosphate levels in a fluid;

FIG. 3 is a schematic side view of another exemplary embodiment of a phosphate level measuring device having two molybdenum electrodes;

FIG. 4 is a schematic side view of one exemplary embodiment of a phosphate level measuring device having one molybdenum electrode;

FIG. 5 is a schematic side view of another exemplary embodiment of a phosphate level measuring device having one molybdenum electrode, the device having an open-cell configuration;

FIG. 6A is a schematic side view of one exemplary embodiment of a configuration of foil-type electrodes in a second chamber of the device of FIG. 1;

FIG. 6B is a schematic side view of one exemplary embodiment of a configuration of wire-type electrodes in a second chamber of the device of FIG. 1;

FIG. 7A is an exploded perspective view of a Double Molybdenum Phosphate Sensor (DMPS) having two Mo electrodes; and

FIG. 7B is an exploded perspective view of a Single Molybdenum Phosphate Sensor (SMPS) having a single Mo electrode.

### GENERAL DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

To the extent features, sides, objects, electrodes, steps, or the like are described as being “first,” “second,” “third,” etc., such numerical ordering is generally arbitrary, and thus such numbering can be interchangeable. For example, in configurations that include two molybdenum electrodes, the electrode closest to the working electrode is often referred to as the second molybdenum electrode, but that electrode may be referred to in the claims as a first molybdenum electrode. Still further, the present disclosure includes some illustrations and descriptions that include prototypes or bench models. A person skilled in the art will recognize how to rely upon the present disclosure to integrate the techniques, systems, devices, and methods related to such prototypes and/or bench models provided for into a product, such as a transportable, phosphate level measuring device.

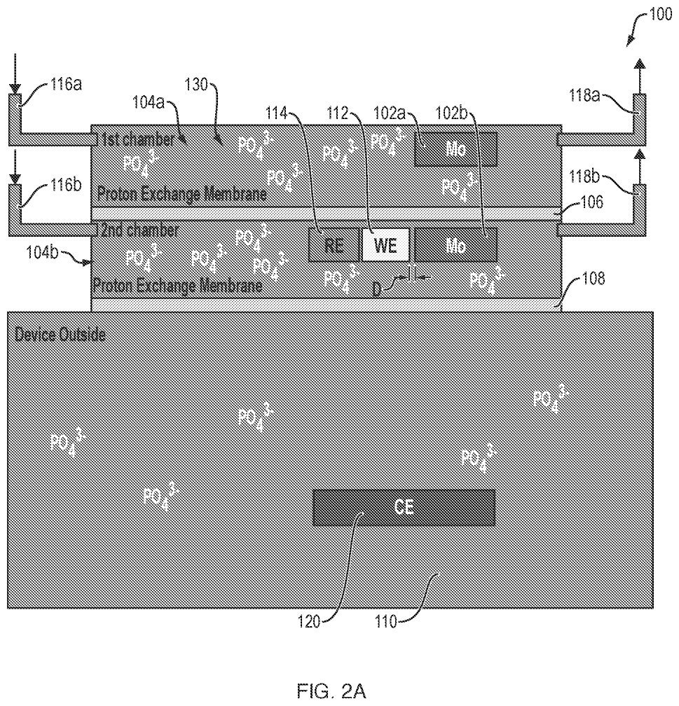

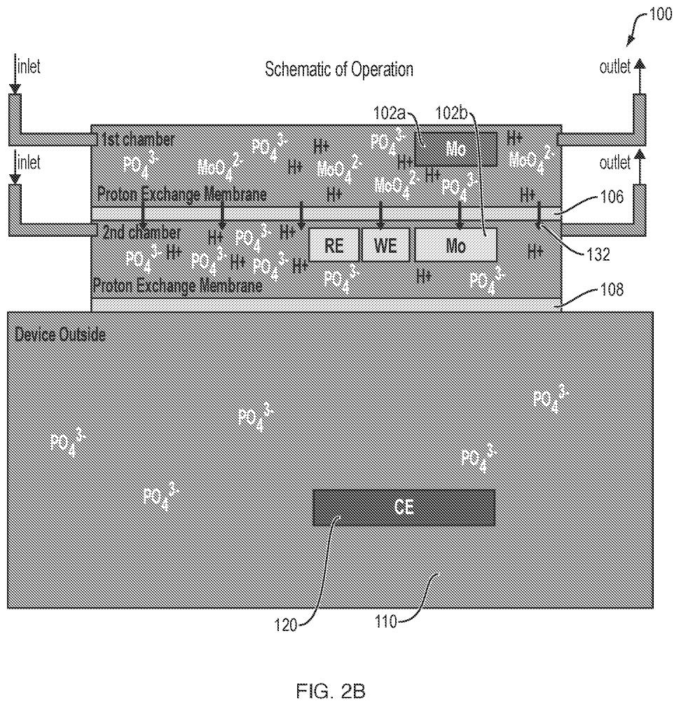

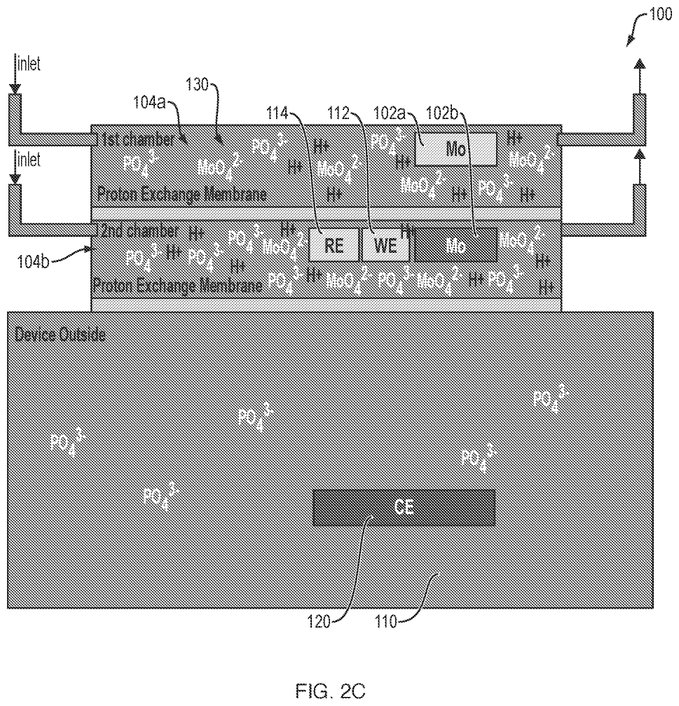

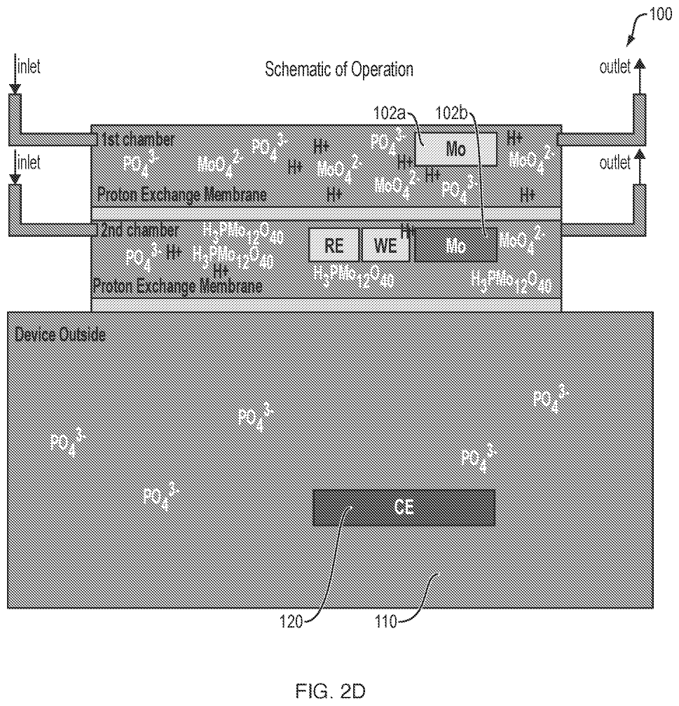

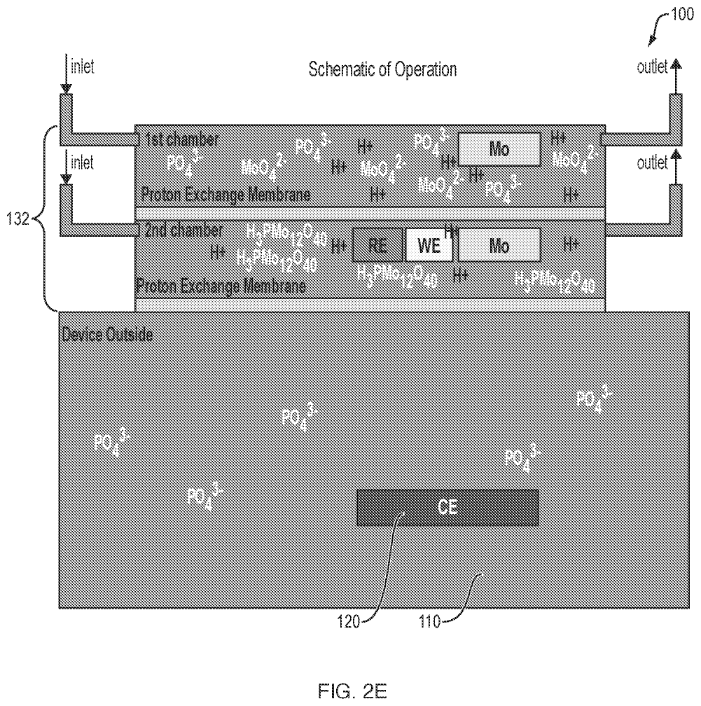

The present disclosure generally provides for small, transportable devices that can be used to make in situ determinations about the level of phosphates in a fluid (e.g., natural water). The fluid is typically a liquid. A molybdenum electrode is used in conjunction with a working electrode, also referred to as a sensing electrode, such that once the molybdenum electrode is oxidized, a reagent is generated that can be measured by an electrochemical set-up to determine how the phosphate level of the fluid being tested. The working electrode is disposed very close to the molybdenum electrode (e.g., within about 100 micrometers or less), and phosphate measurements can be made quickly after the reagents are generated (e.g., within about two minutes, within about thirty seconds, within about ten seconds, depending on the embodiment). Various embodiments of phosphate level determination devices that include one or two molybdenum electrodes are provided. In some instances, the electrodes are disposed in an enclosed chamber through which the fluid being tested is pumped or otherwise driven through, while in other instances an open-cell configuration, i.e., an open chamber, is provided such that the phosphate level determination device can be placed directly in the fluid being measured and operated to make phosphate level determinations. Use of the term “chamber” herein can apply to both an enclosed or closed chamber (e.g., the chambers illustrated in FIGS. 2A-2E, 3, and 4) and an open chamber (e.g., the chamber illustrated in FIG. 5), unless otherwise specified.

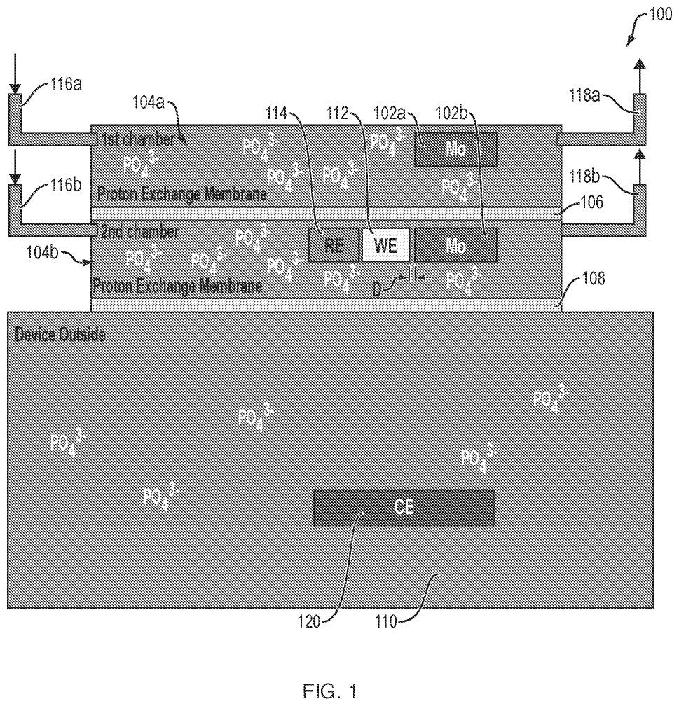

FIG. 1 illustrates a schematic illustration of one embodiment of a phosphate level measuring device 100 that utilizes a first molybdenum (Mo) electrode 102a and a second Mo electrode 102b. The first Mo electrode 102a is at a first location in a first chamber 104a, and the second Mo electrode 102b is at a second location in a second chamber 104b, as shown. The second chamber 104b is separated from the first chamber 104a by a first proton exchange membrane (PEM) 106. As shown, the first and second chambers 104a, 104b are enclosed such that fluid is pumped into and out of the chambers, which is different, for example, than the chamber configuration described with respect to the device of FIG. 5. A person skilled in the art will appreciate that a PEM 106 can be any material that allows protons to pass across it while filtering out other components. The first PEM 106, or other equivalent structure used in conjunction with or in lieu of the first PEM, can generally be configured to transfer protons but not other ions generated in the first chamber to the second chamber. A barrier 108, which can include a second proton exchange membrane (PEM) can be disposed between the second chamber 104b and an outside environment 110, which can include the fluid (e.g., water) being analyzed, ambient air, and so forth. Alternatively, any barrier known to one skilled in the art can be utilized for separating the second chamber 104b from the outside environment 110, including the housing of the second chamber itself without a PEM. Accordingly, non-limiting examples of components that can be used in conjunction with, or in lieu of, the second PEM include other membranes, such as a hydrogel, other structures that can serve as a barrier, and/or other configurations, such as making a narrow flow tunnel to minimize loss.

The second chamber 104b also includes a working electrode 112 and a reference electrode 114, the working electrode 112 being more proximate to the second Mo electrode 102b than the reference electrode 114 is to the second Mo electrode 102b. The working electrode 112 can also be referred to as a sensing electrode. In some embodiments, a distance D between the working electrode 112 and the second Mo electrode 102b, with the distance D being measured between adjacent surfaces of the working electrode 112 and the second Mo electrode 102b, can be approximately in the range of about 10 micrometers to about 1000 micrometers, including some instances where the distance be approximately 100 micrometers or less, or approximately 100 micrometers. In contrast, known devices that includes Mo electrodes and a working electrode are separated into different chambers (see, e.g., “First Deployment and Validation of in Situ Silicate Electrochemical Sensor in Seawater” by Barus, et al., in Marine Science, published Feb. 26, 2018), which thus typically leads to them having a much greater distance between them as compared to the distance between the Mo electrode and working electrode 112 in the present disclosures. Fluid can be passed into the first and second chambers 104a, 104b by respective first and second inlets 116a, 116b and outlets 118a, 118b. As described with respect to FIGS. 2A-2E, protons and other materials can pass between the chambers 104a, 104b and the outside environment 110 via the PEMs 106, 108. As shown, a counter electrode 120 can be disposed within the outside environment 110. Generally, the counter electrode 120 is positioned at a location that is a distance apart from the second Mo electrode 102b and working electrode 112 such that the counter electrode 120 does not consume protons generated by the Mo-working electrode combination.

One non-limiting exemplary embodiment of measuring phosphate levels in a fluid is illustrated by FIGS. 2A-2E. As shown in FIG. 2A, a test solution 130 that includes phosphate ions (e.g., PO₄³⁻) is disposed in the first and second chambers 104a, 104b through the provided inlets. The test solution is introduced into the chambers, and in at least some embodiments can be done such that the test solution 130 fully fills the chambers 104a, 104b. The test solution 130 can be taken from the adjacent fluid being measured, identified in FIG. 1 as the “environment water,” or it can be another fluid that is not necessarily disposed at the location at which the testing is being performed. The former approach can be helpful for instances where field testing on-site is desired.

FIG. 2B illustrates the action of oxidizing the first Mo electrode 102a, and the resulting chemical reactions and migrations that occur. Oxidation of the first Mo electrode 102a can be performed using any techniques known to those skilled in the art, including by supplying voltage to the first Mo electrode 102a. While in the illustrated embodiment the first electrode is an Mo electrode, in other embodiments the first Mo electrode can be replaced by a metal oxidation electrode of a different material(s), such as a tantalum electrode, a titanium electrode, or other electrodes that include metal(s) or metal alloy(s). At the first Mo electrode 102a, the reaction can be depicted as:

Mo(s)+4H₂O→MoO₄²⁻+6H⁺+6e⁻ (1),

while at the counter electrode 120, the reaction can be depicted as:

2H₂O+2e⁻→H₂+2OH⁻ (2).

Protons 132 resulting from oxidation of the first Mo electrode 102a can migrate, in this instance diffuse, across the first PEM 106 and into the second chamber 104b. The protons 132 can help decrease a pH level of the second chamber 104b. For example, the pH level of the second chamber 104b can be reduced to a range approximately between about 0.8 to about 1.2, and in some embodiments the pH level can be reduced to about 1.

As shown in FIG. 2C, the second Mo electrode 102b can also be oxidized, again using any techniques known to those skilled in the art (e.g., supplying voltage to the second Mo electrode), to facilitate anodic dissolution, thereby creating further chemical reactions and the generation of reagent used to measure phosphate levels in the fluid. Similar to the first Mo electrode 102a, the reactions at the second Mo electrode 102b and counter electrode 120 can be depicted as follows:

Mo(s)+4H₂O→MoO₄²⁻+6H⁺+6e⁻ (1), and

2H₂O+2e⁻→H₂+2OH⁻ (2).

The generation of reagent in the second chamber 104b results in the formation of 12-MPA, as shown in FIG. 2D. This reaction can be depicted as follows:

PO₄³⁻+12MoO₄²⁻+27H⁺→H₃PMo₁₂O₄₀+12H₂O (3).

The generation of 12-MPA can then be used to measure phosphate levels in the fluid.

More specifically, an electrochemical sensing set-up 132 can be used to detect the 12-MPA, and thus sense the level of phosphates in the fluid. In the illustrated embodiment in FIG. 2E, the reference electrode 114 and the working electrode 112 are operated using standard techniques for electrochemical measurement to detect the phosphate levels in the fluid. The redox results that are measured in the present embodiment are:

Redox 1: PMo₁₂O₄₀³⁻+2e⁻+2H⁺↔H₂PMo₂^{V}Mo₁₀^{VI}O₄₀³⁻ (4), and

Redox 2: H₂PMo₂^{V}Mo₁₀^{VI}O₄₀³⁻+3e⁻+3H⁺↔H₅PMo₅^{V}Mo₇^{VI}O₄₀³⁻ (5).

To the extent devices and methods exist that can detect 12-MPA in fluid samples, such devices and methods generally require manual sampling, followed by transport and/or storage of that sample before conducting a laboratory-based analysis of the sample. The present embodiments, in contrast, provide reliable, in situ measurement of 12-MPA in fluid samples.

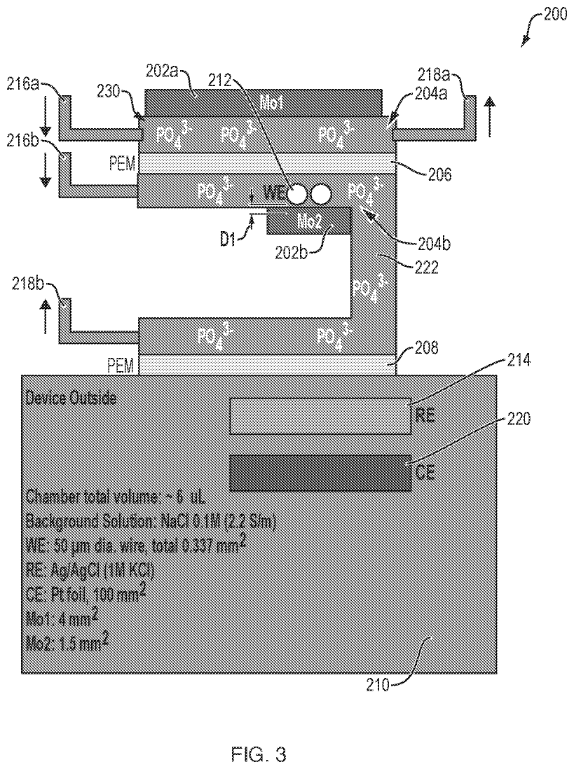

FIG. 3 provides for a phosphate level detection device 200 that includes two electrodes 202a, 202b, sometimes referred to as a double molybdenum electrode device. Except as indicated below and as will be readily appreciated by one having ordinary skill in the art, features of the structure and function of the device 100 can be substantially similar to the device 200 described below, and therefore, a detailed description of these features is omitted here for the sake of brevity. It will be understood that the description of the features of the device 100 can also apply to the device 200 below, unless otherwise noted or unless differences between the two embodiments would be readily understood by a person skilled in the art to operate differently. As shown, the first Mo electrode 202a is at a first location in which at least a portion of the electrode is disposed in a first chamber 204a, and the second Mo electrode 202b is at a second location, in which at least a portion of the electrode can be disposed in a second chamber 204b. In this embodiment, a surface area of the first Mo electrode 202a is significantly larger than a surface area of the second electrode 202b. As shown, a surface area of the first Mo electrode 202a is approximately 4 millimeters² while a surface area of the second Mo electrode 202b is approximately 1.5 millimeters², although other values of these surface areas are possible. For example, in some embodiments a surface area of the first Mo electrode 202a can be approximately in the range of about 2 millimeters² to about 12 millimeters² and a surface area of the second Mo electrode 202b can be approximately in the range of about 0.5 millimeters² to about 8 millimeters². A ratio of a surface area of the first Mo electrode 202a to a surface area of the second Mo electrode can be approximately in the range of about 4:1 to about 1.5:1. Similar to the configuration of FIG. 1, the first and second chambers 204a, 204b can be enclosed such that fluid is pumped into and out of the chambers, which again is different than some other instances, such as the chamber configuration described with respect to the device of FIG. 5. Also similar to the configuration of FIG. 1, the second chamber 204b is again separated from the first chamber 204a by a first PEM 206, and a second PEM 208 can be disposed between the second chamber 204b and an outside environment 210. Other barriers besides a second PEM 208 can be utilized for separating the second chamber 204b from the outside environment 210, including the housing of the second chamber 204b itself without a PEM, as described above with respect to the second PEM of FIG. 1.

The configuration of this device parts differs from the device 100 of FIG. 1 in the set-up of the working, reference, and counter electrodes 212, 214, 220, respectively. As shown, the working electrode 212 is proximate to the second Mo electrode 202b. At least a portion of the working electrode 212 can be disposed in the second chamber. Turning to the reference and counter electrodes 214, 220, in this instance the reference electrode 214 and counter electrode 220 are separate from the device 220. The working electrode 212 is kept near the second Mo electrode 202b so it can quickly capture 12-MPA that is created. In some embodiments, a distance D1 between the working electrode 212 and the second Mo electrode 202b, with the distance D1 being measured between adjacent surfaces of the working electrode 212, e.g., the edge of the circle, and the second Mo electrode 202b, can be approximately in the range of about 10 micrometers to about 1000 micrometers, including some instances where the distance can be approximately 100 micrometers or less, or approximately 100 micrometers. The present embodiment allows a test volume of the device 200, and the distance D1 between the working electrode and the second Mo electrode 202b to be minimized, which can minimize the homogenization time of the 12-MPA from the surface of the second Mo electrode 202b to the working electrode 212. A person skilled in the art will recognize that the device should aim to reach steady state response of 12-MPA as quickly as possible. It has been reported that the formation of 12-MPA in low pH environment ranges approximately from less than about 1 minute to about 5 minutes. The response time, defined as the time to reach the steady state response, can depend, at least in part, on factors such as the geometry of the working electrode 112 and the second molybdenum electrode 102b. Further differences of the configuration of the device 200 are discussed in greater detail below.

In the illustrated embodiment, the working electrode 212 is an approximately 50 micrometer diameter wire, having a total surface area that is approximately 0.337 millimeters², although other diameters, surface areas, structures (i.e., not necessarily a wire), and configurations are possible. For example, in some embodiments a diameter of the wire can be approximately in the range of about 10 micrometers to about 200 micrometers and a total surface can be approximately in the range of about 0.1 millimeters² to about 1.5 millimeters² The configurations of the reference and counter electrodes 214, 220, as well as the background solution, are also provided in FIG. 3, although other configurations and associated values are possible. As discussed above with respect to FIGS. 2A-2E, the counter electrode 220 can be positioned at a location that is far enough away from the Mo electrode 202b and the working electrode 212 such that the counter electrode 220 does not consume protons generated by the Mo-working electrode combination. In the illustrated embodiment, that means the counter electrode 220 is outside of the chamber in which the Mo electrode 202b and the working electrode 212 are disposed, e.g., in the environment 210, as shown, that is outside of the device. The reference electrode 214 can also be placed a distance apart from the Mo-working electrode combination, although the reference electrode 214 does not impact protons in the same manner as the counter electrode 220, and thus its positioning is less impactful in this regard. For example, the positioning of the reference electrode 214 can be inside or outside of the chamber in which the Mo-working electrode is disposed in which a high electrical conductivity background exists, such as for seawater applications. The positioning of the reference electrode 214 can matter more for a low conductivity case, and thus it can be preferable to position the reference electrode 214 in proximity to the working electrode, i.e., as close as possible as understood by a person skilled in the art, for applications that have a low electrical conductivity background.

The second Mo electrode 202b and the working electrode 212 can be disposed proximate, or directly adjacent, to the first PEM 206, which is to say they can be disposed at a top of the diffusion barrier. In some embodiments, to constitute being directly adjacent to the first PEM 206, a distance between the first PEM 206 and at least one of the second Mo electrode 204b and the working electrode 212 is approximately in the range of about 0 micrometers and about 1 millimeter, and in some embodiments it is approximately 500 micrometers. Generally, in accordance with the present disclosures, the closer the second Mo electrode 202b can be to the working electrode 212, the better.

Fluid can be passed into the first and second chambers 204a, 204b by respective first and second inlets 216a, 216b and outlets 218a, 218b. Similar to the configuration of FIG. 1, protons and other materials can pass between the chambers 204a, 204b and the outside environment 210 via the PEMs 206, 208. A total chamber volume of the present device 200 can be significantly smaller than existing analysis devices. For example, a total chamber volume for existing devices can be at least 275 microliters in typical embodiments, while the device of FIG. 3 can have a total chamber volume approximately in the range of about 1 microliter to about 15 microliters, and in some embodiment the volume is about 6 microliters. While in some instances a smaller volume device is preferable for reasons like transportability, in other instances, larger volume devices can also be used in conjunction with the present disclosures. Thus, the electrode configurations provided for herein can be used in devices having a total chamber volume of at least about 25 microliters, at least about 50 microliters, at least about 100 microliters, at least about 150 microliters, at least about 200 microliters, at least about 250 microliters, and at least about 275 microliters or greater, e.g., 100 microliters, 1 milliliter. As the volume of the chamber(s) increases, so too can a size (e.g., surface area) of the electrodes used in conjunction with the same.

It will be appreciated that when using the instant device 200 on the surface water of a pond or lake, which typically has a conductivity approximately 100 times smaller than that of seawater, up to 100 times more energy for the oxygenation of molybdenum may be used. However, if the decrease in the energy consumption coming from the decrease of the test volume is significant enough, the increase in the energy consumption caused by using the lower conductivity might not be significant enough compared to the total energy consumption of the device. Moreover, the decrease of the test volume per measurement makes the device more portable and/or extends the working time of the device. Further, the complexation time of 12-MPA can be greatly reduced as the small volume can facilitate the homogenization of molybdate ions. Under these assumptions, the device can be applied to various types of natural water without a huge disadvantage.

A method of using the double molybdenum detection device 200 can be similar to the method described above with respect to FIGS. 2A-2E, although the provided configuration of the double molybdenum detection device can provide for enhanced methods and results. The reactions and redox results can be the same, and thus are not repeated when describing the method of using the device of FIG. 3.

After fluid 230 enters the chambers, the first Mo electrode 202a can be oxidized. The oxidation can occur for a time period approximately in the range of about 20 seconds to about 60 seconds (e.g., 20 seconds, 40 seconds, 60 seconds), although other values of time less or greater than that are possible. The current density supplied to the first Mo electrode 202a can be approximately in the range of about 0.1 milliampere/millimeters² to about 2 milliamperes/millimeters², for example about 0.5 milliamperes/millimeters², although other values of current density less or greater than that are possible. An oxidation time period and a current density can be linked such that a small current density can be used in conjunction with a longer oxidation time period or a larger current density can be used in conjunction with a shorter oxidation time period. Further, these values can also depend on the size and shape of the components of the device and/or the device itself. Accordingly, as a volume of the chamber 204a and/or a surface area of the first Mo electrode changes 202a, so too can the oxidation time period and/or the current densities to achieve desirable results. After oxidation of the first Mo electrode 202a occurs, protons subsequently migrate (e.g., diffuse) across the first PEM 206 and into the second chamber 204b, which can decrease a pH level in the second chamber (e.g., to a value approximately in the range of about 0.8 to about 1.2, including to about 1, as described above with respect to FIGS. 2A-2E).

The second Mo electrode 204b can then be oxidized. This can occur for a time period approximately in the range of about 1 second to about 20 seconds (e.g., 2 seconds), although other values of time less or greater than that are possible. The current density supplied to the second Mo electrode 202b can be approximately in the range of about 0.001 milliamperes/millimeters² to about 1 milliampere/millimeters², for example about 0.05 milliamperes/millimeters², although other values of current density less or greater than that are possible. That is, similar to the first Mo electrode 202a, an oxidation time period and a current density for the second Mo electrode 202b can be linked such that a small current density can be used in conjunction with a longer oxidation time period or a larger current density can be used in conjunction with a shorter oxidation time period. Further, these values can also depend on the size and shape of the components of the device and/or the device itself. Accordingly, as a volume of the chamber 204b and/or a surface area of the second Mo electrode changes 202b, so too can the oxidation time period and/or the current densities to achieve desirable results. In at least some embodiments, after approximately two seconds, 12-MPA formation can begin.

Moreover, the instant configuration of device 200 differs from the device 100 in that the device 200 includes a diffusion barrier 222 disposed between the first chamber 104a and the second chamber 104b. As shown, the diffusion barrier 222 can be in the form of a vertical column that leads from the first chamber 204a to the second chamber 204b, though it will be appreciated that a size and shape of the diffusion barrier 222 can vary based, at least in part, on the shape of the device, the substances used, and/or other design parameters known to one skilled in the art. For example, during oxidation of the first Mo electrode 202a, the diffusion barrier delays the diffusion of protons from the first chamber 204a to the outside environment 210 to create a diffusion gradient along the second chamber 204b, with the first chamber having the lowest pH and gradually increasing through the diffusion barrier 222 and through the second chamber 204b, a bottom of the second chamber 204b having the highest pH values. Having such a gradient reduces the time and amount of energy used to lower the chamber pH as it provides a local area with a pH that is low enough for 12-MPA formation rather than decreasing the pH of an entire volume of the chamber. As a result, the working electrode 212 and the second molybdenum electrode 202b are placed in the upper part of the second chamber 204b so that they can experience the lowest possible pH along the chamber.

Use of two separate molybdenum oxidations allows for control of the pH of the reaction environment because it can determine the efficiency of the formation of both 12-MPA and 12-MSA. 12-MSA, 12-molybdo-silicic-acid, can be a major interference molecule in the detection of 12-MPA, which is formed between silicate, molybdates, and protons. Optimization of the formation of 12-MPA to maximize the amount of 12-MPA while minimizing that of 12-MSA, which can avoid the interference from silicate ion, can occur when there exists approximately 70 times excess molar concentration of protons than that of molybdate. Optimization of the oxidation of the first molybdenum electrode 102a is therefore desired to realize optimum conditions in the second chamber 104b.

Testing of the device 200 illustrated in FIG. 3 yields performances that far exceed that of existing devices. For example, while existing devices that include molybdenum can typically consume about 9 milligrams per measurement, a molybdenum consumption level of the present device is approximately in the range of about 0.05 milligrams per measurement to about 1 milligrams per measurement, and in some embodiments it is approximately 0.08 milligrams per measurement or less. By way of further example, while existing devices that include molybdenum have an energy consumption for molybdenum oxidation of about 18 Joules per measurement, an energy consumption level for molybdenum oxidation of the present device is approximately in the range of about 0.5 Joules per measurement to about 5 Joules per measurement, and in some embodiments it is approximately 0.8 Joules per measurement or less. In view of the second Mo electrode 202b having an exposed surface area of about 4 millimeters², the resulting energy per measurement value can be defined as approximately 0.2 Joules or less of energy for each 1 millimeter² of exposed molybdenum electrode surface area consumed per measurement. A person skilled in the art, in view of the present disclosures, will appreciate that these values may change as other parameters change, including but not limited to oxidation time periods, current densities, volumes of chamber(s), and/or surface areas of electrode(s). Nevertheless, the material (e.g., molybdenum) consumption levels and energy consumption levels represent stark improvements over existing methods and devices, regardless of size, but particularly in view of the small size at which these values can be achieved in view of the present disclosures.

The detection time and range is also vastly improved by the present disclosures. For example, while existing devices for phosphate level determinations can take about 70 minutes to perform their analysis without stirring the fluid (i.e., the 12-MPA) and about 5 minutes with stirring the fluid (such as by using a pump, as described above), the present device can detect phosphate levels (i.e., a phosphate level detection time) approximately in the range of about 1 minute to about 10 minutes without stirring the fluid (i.e., the 12-MPA), and in some embodiments about 2 minutes or less without stirring the fluid. These times may be even faster if the fluid is stirred. Still further, a linear detection range of existing devices can be approximately in the range of about 0.1 μM to about 1 μM or about 0.25 μM to about 4 μM, while the linear detection range of the present device 200 can be approximately in the range of about 1 μM to about 25 μM. Other linear detection ranges, including those that exceed about 25 μM, may also be possible in view of the present disclosures.

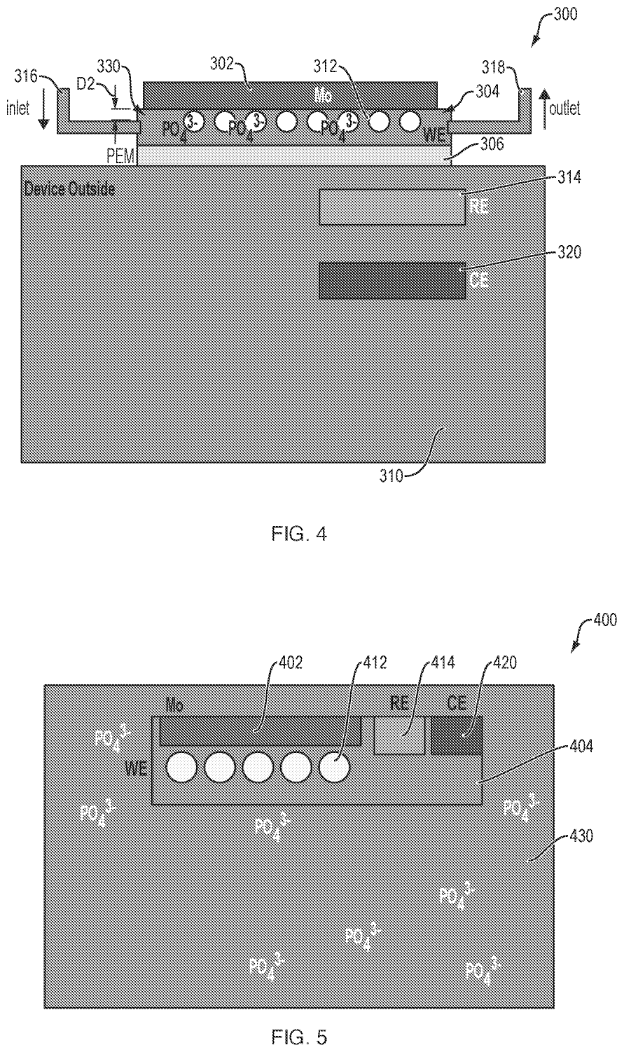

FIG. 4 provides for a phosphate level detection device 300 that includes a single electrode 302, sometimes referred to as a single molybdenum electrode device. As shown, an Mo electrode 302 can be at a location in which at least a portion of the electrode is disposed in a chamber 304. Only one chamber is provided, with that chamber being more akin to the second chamber 204b than the first chamber 204a of the device of FIG. 3. As shown, the first chamber 304 is enclosed such that fluid 330 is pumped into and out of it, which is different than the chamber configuration described with respect to the device of FIG. 5. As also shown, the Mo electrode 302 has a surface area that is approximately 4 millimeters², although other sizes and configurations are possible, such as approximately in the range of about 1 millimeter²to about 15 millimeters². The chamber 304 is separated from the outside environment 310 by a PEM 306, although other barriers besides a PEM can be utilized, including the housing of the chamber 304 itself without a PEM, as described above with respect to the second PEM 308 of FIG. 1.

Similar to the device of FIG. 3, the working electrode 312 is proximate to the Mo electrode 302. At least a portion of the working electrode 312 can be disposed in the chamber 304. Also similar to the device of FIG. 3, the reference and counter electrodes 314, 320 are separate from the device. Again, the working electrode 312 is kept near the Mo electrode 302 so it can quickly capture 12-MPA that can be created. In some embodiments, a distance D2 between the working electrode 312 and the Mo electrode 302, with the distance D2 being measured between adjacent surfaces of the working electrode 312, e.g., the edge of the circle, and the second Mo electrode 302b, can be approximately in the range of about 10 micrometers to about 1000 micrometers, including some instances where the distance be approximately 100 micrometers or less, or approximately 100 micrometers. In some embodiments, the working electrode 312 and the Mo electrode 302 can be closer, e.g., have smaller values of the distance D2, than the electrode and the working electrode 212 in device 200, which can allow the device 300 to function having a single electrode 302. The present embodiment allows a test volume of the device 300, and the distance between the working electrode and Mo electrode to be minimized, which can minimize the homogenization time of the 12-MPA from the surface of the Mo electrode 304 to the working electrode 312. The working electrode 312 of the present device 300 is different from that of the working electrode 212 in FIG. 3 in that a total surface area is significantly larger. While in the illustrated embodiment the working electrode 312 is an approximately 50 micrometer diameter wire, a total surface area is approximately 1.13 mm². Again, other diameters, surface areas, structures (i.e., not necessarily a wire), and configurations are possible. For example, in some embodiments a diameter of the wire can be approximately in the range of about 10 micrometers to about 200 micrometers and a total surface can be approximately in the range of about 0.5 millimeters² to about 5 millimeters² The configurations of the reference and counter electrodes 314, 320, as well as the background solution, are akin to those from FIG. 3, although other configurations and associated values are possible. This configuration is generally considered simpler than that of the device of FIG. 3 because there are fewer components (e.g., chambers).

Fluid 330 can be passed into the chamber 304 by the illustrated inlet 316 and outlet 318. A total chamber volume of the present device 300 can be significantly smaller than existing analysis devices. For example, a total chamber volume for existing devices can be at least 275 microliters in typical embodiments, while the device 300 of FIG. 4 can have a total chamber volume approximately in the range of about 0.5 microliters to about 50 microliters, and in some embodiment the volume is about 1.5 microliters. The total chamber volume can be larger as well, as referenced above, with other components associated with the device (e.g., surface area(s) of electrode(s)) also having the possibility of being larger as well. In an open-cell configuration, as at least described below with respect to FIG. 5, the volume can essentially be infinite.

A method of using this single molybdenum detection device can arguably be even simpler than the device 200 of FIG. 3. This is at least because with only one Mo electrode 302, oxidation can be limited to a single action for one electrode. Thus, the method can involve oxidizing the Mo electrode 302 to form 12-MPA and running SWV sweeps at various points in time during the detection process.

There are a number of operating conditions that separate the device 300 from the device 200, which can allow the device 200 to outperform both existing devices and that of device 200. For example, an amount of oxidation of Mo in the device 300 can be sufficiently high to supply a sufficient amount of protons for 12-MPA formation, which is much higher than the oxidation of the second Mo electrode 204b in the device 200. In fact, the device 300 can oxidize an amount of molybdenum that is so large so as to achieve both a PH reduction due to the proton generation and a formation of the 12-MPA in presence of orthophosphate ions in the same chamber and at the same time. The device 200 instead uses a smaller current than that of device 300, with the current being passed through the second Mo electrode because the function of the electrode is only towards the formation of 12-MPA. The pH is being regulated by a separate molybdenum oxidation chamber in the configuration discussed with respect to the device 200.

Testing of the device 300 illustrated in FIG. 4 yields performances that far exceed that of existing devices, and even the performances of the device of FIG. 3. For example, while existing devices that include molybdenum can typically consume about 9 milligrams per measurement, a molybdenum consumption level of the present device is approximately in the range of about 0.0005 milligrams per measurement to about 0.01 milligrams per measurement, and in some embodiments it is approximately 0.0008 milligrams per measurement or less. By way of further example, while existing devices that include molybdenum have an energy consumption for molybdenum oxidation of about 18 Joules per measurement, an energy consumption level for molybdenum oxidation of the present device is approximately in the range of about 0.0005 Joules per measurement to about 0.1 Joules per measurement, and in some embodiments it is approximately 0.001 Joules per measurement or less. In view of the Mo electrode having an exposed surface area of about 4 millimeters², the resulting energy per measurement value can be defined as approximately 0.00025 Joules or less of energy for each 1 millimeter² of exposed molybdenum electrode surface area consumed per measurement. A person skilled in the art, in view of the present disclosures, will appreciate that these values may change as other parameters change, including but not limited to oxidation time periods, current densities, volumes of chamber(s), and/or surface areas of electrode(s). For example, as a surface area(s) of an electrode(s) is increased, so too can the volume of the chamber 304 in which the electrode(s) 304 is disposed. Nevertheless, the material (e.g., molybdenum) consumption levels and energy consumption levels represent stark improvements over existing methods and devices, regardless of size, but particularly in view of the small size at which these values can be achieved in view of the present disclosures. Further, because the single Mo electrode 302 approach as provided only includes a single chamber 304, parameters such as material (e.g., molybdenum) consumption, energy consumption, and current density are typically prevented from going too high to avoid possible silicate interference. The pH level should be maintained in a desired range to prevent silicate from forming 12-MSA such that optimal Mo oxidation conditions only result in 12-MPA formation and not 12-MSA formation.

The detection time is also vastly improved by the present disclosures. For example, while existing devices for phosphate level determinations can take about 70 minutes to perform their analysis without stirring the fluid (i.e., the 12-MPA) and about 5 minutes with stirring the fluid (such as by using a pump, as described above), the present device can detect phosphate levels (i.e., a phosphate level detection time) approximately in the range of about 10 seconds to about 2 minutes without stirring the fluid (i.e., the 12-MPA), and in some embodiments about 30 seconds or less without stirring the fluid. These times may be even faster if the fluid is stirred. The device can have a non-linear relation, as opposed to a linear detection range as described above. While linear detection ranges can sometimes be preferable, non-linear relations will typically also work for the intended purposes of the present disclosure.

FIG. 5 provides for another phosphate level detection device 400 that includes a single electrode 402, again sometimes referred to as a single molybdenum electrode device. This configuration differs from the configuration of the device of FIG. 4 in least that the configuration of the device in FIG. 5 has an open-cell arrangement—no additional set-up is required to pump the fluid 430 being tested into and out of the device.

As shown, each of an Mo electrode 402, working electrode 412, reference electrode 414, and counter electrode 420 are provided as part of a device that is associated with the fluid to be tested. Rather than an enclosed chamber, as described in earlier embodiments, the device includes an open chamber 404, sometimes referred to as a mounting component or mounting plate, with which the electrodes are coupled or otherwise associated. The illustration in FIG. 5 is a side view, but from a top view, a bottom and top can include openings such that the housing has an open-cell configuration. As a result, the volume of the device 400 can be approximately infinite. The mounting plates 404 provide for a way by which the electrodes—the Mo electrode 402, the working electrode 412, the counter electrode 420, and the reference electrode 414 as illustrated—can be disposed within the fluid being tested. More specifically, at least a portion of the various electrodes can be disposed within the chamber 404. For an open-cell configuration like that of FIG. 5, being “disposed within” the chamber can mean within the bounds of walls of a housing that form the chamber, or within the bounds of other structure(s) that form or otherwise define the chamber. The configuration of the device of FIG. 5 allows it such that no pump or equivalent component is needed to move the liquid to a location where the analysis is to occur. Accordingly, at least because this configuration does not require any additional set-up to run the test, such as a pump, it can be considered an even more simple device 400 than the device 300 of FIG. 4.

In the illustrated embodiment, the Mo electrode 402 has a surface area that is approximately 4 millimeters², and the working electrode 412 is an approximately 50 micrometer diameter wire having a total surface area that is approximately 1.13 millimeters², although other sizes and configurations are possible. For example, in some embodiments a diameter of the wire can be approximately in the range of about 10 micrometers to about 200 micrometers and a total surface can be approximately in the range of about 0.5 millimeters² to about 5 millimeters² The configurations of the reference and counter electrodes 414, 420, as well as the background solution, are akin to those from FIGS. 3 and 4, although other configurations and associated values are also possible.

A method of using this single molybdenum detection device 400 can arguably be even simpler than the devices 200, 300 of FIGS. 3 and 4. This is at least because no oxidation steps are needed, and no pumping of fluid into and out of a chamber is used either. Thus, the method can merely entail disposing the device in the fluid to be tested and detecting a phosphate level.

Testing of the device 400 illustrated in FIG. 5 yields performances that far exceed that of existing devices, and even the performances of the device 200 of FIG. 3. For example, while existing devices that include molybdenum can typically consume about 9 milligrams per measurement, molybdenum consumption level of the present device is approximately in the range of about 0.0005 milligrams per measurement to about 0.01 milligrams per measurement, and in some embodiments it is approximately 0.0008 milligrams per measurement or less. By way of further example, while existing devices that include molybdenum have an energy consumption for molybdenum oxidation of about 18 Joules per measurement, an energy consumption level for molybdenum oxidation of the present device is approximately in the range of about 0.0005 Joules per measurement to about 0.1 Joules per measurement, and in some embodiments it is approximately 0.001 Joules per measurement or less.

The detection time is also vastly improved by the present disclosures. For example, while existing devices for phosphate level determinations can take about 70 minutes to perform their analysis without stirring the fluid (i.e., the 12-MPA) and about 5 minutes with stirring the fluid (such as by using a pump, as described above), the present device can detect phosphate levels (i.e., a phosphate level detection time) approximately in the range of about 1 second to about 1 minute without stirring the fluid (i.e., the 12-MPA), and in some embodiments about 10 seconds or less without stirring the fluid. These times may be even faster if the fluid is stirred.

Although the embodiments described herein are sometimes referred to as double or single molybdenum detection devices, such devices can include additional electrodes without departing from the spirit of the present disclosure.

A person skilled in the art will recognize that a benefit of the present disclosures is the minimal amount of components that are needed to make phosphate level detections, and the resulting size of the devices that allows them to be transportable and used in situ directly on-site. An important aspect of the design strategy of the device 100 is to minimize a thickness of the chambers 104a, 104b while maximizing the cross-sectional area of each layer. The thin layers of the chambers 104a, 104b will compensate the increase in ohmic resistance caused by the volume reduction, and decrease the diffusion length between the first and second chambers 104a, 104b, resulting in faster transfer/diffusion of protons across the first PEM 106. Moreover, performance is increased when the surface area of the molybdenum electrode in the chambers is maximized. For the molybdenum electrode 102a in the first chamber 104a, the larger surface area will generate more protons under the same current density, which can result in less time needed to reach the desired pH. Similarly, the larger surface area of the second molybdenum electrode 102b can facilitate the homogenization of molybdate ions for the faster formation of 12-MPA. Still further, a smaller distance between the second molybdenum electrode 102b and the working electrode 112 decreases the diffusion length of the 12-MPA reducing the response time. It will be appreciated that although this is being discussed with respect to the embodiment of the device 100, these concepts can apply to all of the devices 200, 300, 400 of the present disclosure.

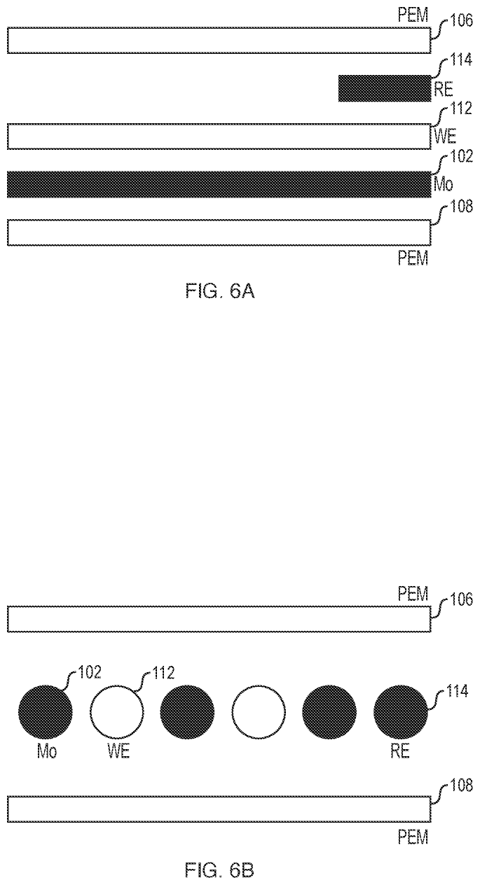

FIGS. 6A-6B illustrate exemplary embodiments of possible configurations of the electrodes in the second chamber 104b. As noted above, larger surface areas of the electrodes as well as the working electrode 112 can be beneficial as more 12-MPA will be electrochemically detected when the surface area is larger, thereby resulting in better sensitivity. Foil-type electrodes, as shown in FIG. 6A, can maximize the exposed surface area, however, the Ohmic resistance in the second chamber 104b can be increased by blocking the path for transfer ions. Moreover, the layout of FIG. 6A can complicate installation of the electrodes (reference electrode 114, working electrode 112, and electrode 102 disposed between the first and second PEM 106, 108) without making contact between one another as the thickness of the chamber 104b decreases, such as in when the device 100 is reduced in size for increased portability.

FIG. 6B illustrates wire-type electrodes being disposed in the second chamber 104b. As shown, wire-type electrodes can allow for orientations of multiple working electrodes 112 and molybdenum electrodes 102, the homogenization of molybdate ions during the formation of 12-MPA can be maximized while the distance between the molybdenum electrodes 102 and working electrodes 112 is minimized for faster response.

Each of the devices 100, 200, 300, 400 discussed herein can be portable, autonomous, and capable of in situ measurements.

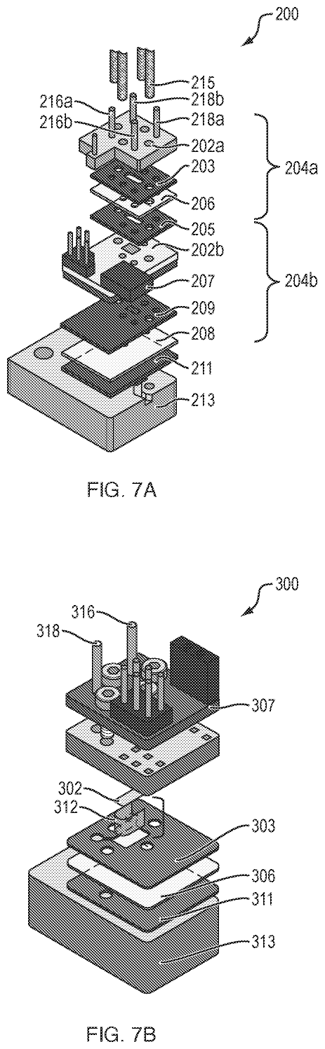

FIG. 7A illustrates an exemplary embodiment of a Double Molybdenum Phosphate Sensor (DMPS) that is discussed schematically in FIG. 3. As shown, the device can comprise multiple layers stacked together with the components discussed above. For example, as discussed above, the DMPS can include two chambers separate by the first PEM 206. The first chamber 204a can include a first layer having the first Mo electrode 202a and a second layer having a first chamber conduit 203. The first Mo electrode 202a can be installed at the bottom of the first layer, which can be exposed to a flow of fluid(s) (i.e., “a fluid flow”) through a channel in the first chamber conduit 203. The inlets 216a, 216b and outlets 218a, 218b of the device 200 can extend from the first layer. A series of screws 215 can be received in the first layer and extend through the device to fix the layers relative to one another. A person skilled in the art will appreciate other coupling mechanisms that can be used in lieu of or in addition to screws to fix the layers relative to one another, such as one or more of nails, staples, bolts, shanks, adhesives, and so forth.

Oxidation of the first Mo electrode 202a, with the first Mo electrode 202a being installed at the bottom of the first layer in the first chamber 204a can provide protons through the third layer having the first PEM 206 into the second chamber 204b. The second chamber 204b, as shown, can include a fourth layer having a second chamber upper conduit 205, a fifth layer having the second Mo electrode 202b, a sixth layer having a connection board 207, and a seventh layer having a second chamber lower conduit 209. The device 200 can further include an eighth layer having a second PEM 208, a ninth layer having a sealing gasket 211, and a tenth layer including a separator 213. In some embodiments, the second chamber 204b can include a gold wire electrode as a working electrode. The gold wire electrode can be positioned on top of the second Mo electrode 202b without contacting the second Mo electrode 202b. Additional details related to a gold wire electrode are described below with respect to the device 300 of FIG. 7B, and are applicable for a gold wire electrode used in other embodiments, such as the device 200 of FIG. 7A. It will be appreciated that in some embodiments the device 200 may not function without the working electrode. As noted above, in some embodiments, the gold wire electrode and the second Mo electrode 202b can be positioned in the upper part of the second chamber 204b so that they can experience the lowest possible pH along the chamber. These electrodes can be exposed to a fluid flowing through a fluid channel in the second chamber upper conduit 205. The second Mo electrode 202b in the first layer can be oxidized to produce molybdate ions which reacts with phosphate ions to form 12-MPA, which can be detected by the gold electrode. It will be appreciated that the first chamber conduit 203, second chamber upper conduit 205, and second chamber lower conduit 209 can function as fluid chambers through which the sample fluid with phosphate ions passes, as well as the sealing material in between each layer. A person skilled in the art will recognize that the above-described embodiment is merely exemplary and one or more of the layers can be omitted, replaced, and/or juxtaposed when forming the DMPS.

FIG. 7B illustrates an exemplary embodiment of a Single Molybdenum Phosphate Sensor (SMPS). As shown, the device can be made up of multiple layers stacked together with the components discussed above. For example, as discussed above, the SMPS can include a first layer having a connection board 307 and a second layer having the Mo electrode 302. The Mo electrode 302 can be installed under the second layer, as shown. The remaining layers of the device 300 can include a third layer having a chamber conduit 303, a fourth layer having a PEM 306, a fifth layer having a sealing gasket 311, and a sixth layer having a separator 313. The Mo electrode 302 can be exposed to a flow of fluid(s) (i.e., “a fluid flow”) through a channel in the chamber conduit 303. The inlet 316 and outlet 318 of the device 300 can extend from the first layer. In some embodiments, the device 300 can include a gold wire electrode 312. Installation of the gold wire electrode 312 along with the Mo electrode 302 can allow the device 300 to function as a sensor. The Mo electrode 302 and the gold wire electrode 312 can be installed at the bottom of the second layer. As in the above, the gold wire electrode 312 can be positioned on top of the Mo electrode 302 without contacting the Mo electrode 302. It will be appreciated that in some embodiments the device 300 may not function without the working electrode. These electrodes can be exposed to a fluid flowing through a fluid channel in the chamber conduit 303. The Mo electrode 302 can be oxidized to produce molybdate ions and protons, which reacts with phosphate ions to form 12-MPA, which can be detected by the gold electrode. It will be appreciated that the chamber conduit 303 can function as fluid chambers through which the sample fluid with phosphate ions passes, as well as the sealing material in between each layer. A person skilled in the art will recognize that the above-described embodiment is merely exemplary and one or more of the layers can be omitted, replaced, and/or juxtaposed when forming the SMPS. Moreover, one or more features discussed with respect to the DMPS can also be applied to the SMPS and vice versa and are omitted herein for the sake of brevity.

Examples of the above-described embodiments can include the following:

1. A phosphate level detection device, comprising:
a first chamber;
a first molybdenum electrode at least partially disposed within the first chamber; and
a working electrode at least partially disposed within the first chamber, the working electrode being positioned within about 100 micrometers of the first molybdenum electrode, wherein the device is configured such that oxidation of the first molybdenum electrode in the presence of a fluid that includes phosphate ions results in the formation of a 12-molybdophosphoric acid.
   2. The phosphate level detection device of claim 1, further comprising:
a second chamber, each of the first chamber and the second chamber being enclosed chambers;
a second molybdenum electrode at least partially disposed in the second chamber; and
a proton exchange membrane disposed between the first chamber and the second chamber,
wherein the device is configured such that oxidation of the second molybdenum electrode in the presence of a fluid results in protons migrating from the second chamber, across the proton exchange membrane, and to the first chamber to reduce a pH level of fluid disposed in the first chamber.
   3. The phosphate level detection device of claim 2, wherein the device is configured such that the protons that migrate from the second chamber, across the proton exchange membrane, and to the first chamber reduce the pH level to a range of about 0.8 to about 1.2.
   4. The phosphate level detection device of claim 2 or claim 3, wherein the first molybdenum electrode and the working electrode are disposed directly adjacent to the proton exchange membrane.
   5. The phosphate level detection device of any of claims 1 to 4, further comprising at least one of:
a reference electrode; or
a counter electrode,
wherein the at least one of the reference electrode or the counter electrode, in conjunction with the working electrode, are configured to make electrochemical determinations of a phosphate level of a fluid disposed in the first chamber when the first molybdenum electrode is oxidized.
   6. The phosphate level detection device of claim 1, further comprising:
a reference electrode; and
a counter electrode;
wherein the first chamber is an open chamber and each of the first molybdenum electrode, the working electrode, the reference electrode, and the counter electrode are at least partially disposed within bounds defined by walls of the first chamber, the device being configured to operate in an open-cell configuration.
   7. The phosphate level detection device of any of claims 1 to 5, wherein the device has a total chamber volume of about 6 microliters or less.
   8. The phosphate level detection device of claim 1 or claim 5, wherein the device has a total chamber volume of about 1.5 microliters or less.
   9. The phosphate level detection device of any of claims 1 to 8, wherein a molybdenum consumption level is approximately 0.08 milligrams or less per measurement.
   10. The phosphate level detection device of any of claim 1, claim 5, claim 6, or claim 8, wherein a molybdenum consumption level is approximately 0.0008 milligrams or less per measurement.
   11. The phosphate level detection device of any of claims 1 to 10, wherein an energy consumption level for oxidation of the first molybdenum electrode is approximately 0.2 Joules or less per measurement for each 1 millimeter² of exposed first molybdenum electrode surface area.
   12. The phosphate level detection device of any of claim 1, claim 5, claim 6, claim 8, or claim 10, wherein an energy consumption level for oxidation of the first molybdenum electrode is approximately 0.00025 Joules or less per measurement for each 1 millimeter² of exposed first molybdenum electrode surface area.
   13. The phosphate level detection device of any of claims 1 to 12, wherein a phosphate level detection time of the device is approximately two minutes or less in the absence of stirring the 12-molybdophosphoric acid.
   14. The phosphate level detection device of any of claim 1, claim 5, claim 6, claim 8, claim 10, or claim 12, wherein a phosphate level detection time of the device is approximately 30 seconds or less in the absence of stirring the 12-molydophosphoric acid.
   15. The phosphate level detection device of any of claim 1, claim 5, claim 6, claim 10, or claim 12, wherein a phosphate level detection time of the device is approximately 10 seconds or less in the absence of stirring the 12-molydophosphoric acid.
   16. A method for determining phosphate levels in a fluid, comprising:
oxidizing a first molybdenum electrode that is at least partially disposed in a first chamber of a phosphate level detection device to form a 12-molybdophosphoric acid from a fluid having phosphate ions disposed therein; and
determining a level of phosphate ions present in the fluid from which the 12-molybdophosphoric acid is formed using an electrochemical set-up, the electrochemical set-up including a working electrode disposed within about 100 micrometers of the first molybdenum electrode.
   17. The method of claim 16, further comprising:
causing the fluid having phosphate ions disposed therein to enter the first chamber,
wherein the fluid comes from a fluid source that is at a location at which the oxidizing and determining actions are performed such that determining a level of phosphate ions present in the fluid from which the 12-molybdophosphoric acid is formed occurs in situ.
   18. The method of claim 16 or claim 17, further comprising:
oxidizing a second molybdenum electrode that is at least partially disposed in a second chamber to cause protons to migrate from the second chamber, to the first chamber, to reduce a pH level of the fluid disposed in the first chamber,
wherein each of the first and second chambers are enclosed.
   19. The method of claim 18, further comprising:
causing the fluid having phosphate ions disposed therein to enter the second chamber,
wherein the fluid comes from a fluid source that is at a location at which the oxidizing and determining actions are performed such that determining a level of phosphate ions present in the fluid from which the 12-molybdophosphoric acid is formed occurs in situ.
   20. The method of claim 18 or claim 19, wherein migration of the protons from the second chamber to the first chamber reduces the pH level of the fluid disposed in the first chamber to a range of about 0.8 to about 1.2.
   21. The method of any of claims 18 to 20, wherein the first molybdenum electrode and the working electrode are disposed directly adjacent to the second chamber.
   22. The method of any of claims 16 to 21, wherein a total chamber volume of the phosphate level detection device is about 6 microliters or less.
   23. The method of claim 16 or claim 17, wherein a total chamber volume of the phosphate level detection device is about 1.5 microliters or less.
   24. The method of any of claims 16 to 23, wherein approximately 0.08 milligrams of molybdenum or less is consumed per measurement.
   25. The method of any one of claim 16, claim 17, or claim 23, wherein approximately 0.0008 milligrams of molybdenum or less is consumed per measurement.
   26. The method of any of claims 16 to 25, wherein approximately 0.2 Joules or less of energy for each 1 millimeter² of exposed first molybdenum electrode surface area is consumed per measurement.
   27. The method of any one of claim 16, claim 17, claim 23, or claim 25, wherein approximately 0.00025 Joules or less of energy for each 1 millimeter² of exposed first molybdenum electrode surface area is consumed per measurement.
   28. The method of any of claims 16 to 27, wherein determining a level of phosphate ions present in the fluid from which the 12-molybdophosphoric acid is formed using an electrochemical set-up consumes approximately two minutes or less of time in the absence of stirring the 12-molybdophosphoric acid.
   29. The method of any one of claim 16, claim 17, claim 23, claim 25, or claim 27, wherein determining a level of phosphate ions present in the fluid from which the 12-molybdophosphoric acid is formed using an electrochemical set-up consumes approximately thirty seconds or less of time in the absence of stirring the 12-molybdophosphoric acid.
   30. The method of claim 16, wherein determining a level of phosphate ions present in the fluid from which the 12-molybdophosphoric acid is formed using an electrochemical set-up consumes approximately ten seconds or less of time in the absence of stirring the 12-molybdophosphoric acid.
   31. A method for determining phosphate levels in a fluid, comprising:
sampling a fluid having phosphate ions disposed therein from a fluid source;
generating a reagent from the fluid having phosphate ions disposed therein due to anodic dissolution of molybdenum metal; and
determining a level of phosphate ions in the fluid from which the reagent is generated,
wherein the actions of sampling, generating, and determining are all performed at the location of the fluid source such that determining a level of phosphate ions in the fluid from which the reagent is generated occurs in situ.
   32. The method of claim 31, wherein generating a reagent from the fluid having phosphate ions disposed therein due to anodic dissolution of molybdenum metal further comprises:
oxidizing a first molybdenum electrode that includes the molybdenum metal that is dissolved.
   33. The method of claim 32, wherein a working electrode used in conjunction with determining a level of phosphate ions in the fluid from which the reagent is generated is disposed within about 100 micrometers of the first molybdenum electrode.
   34. The method of claim 32 or claim 33, wherein the first molybdenum electrode is disposed in a first enclosed chamber of a phosphate level determination device, the method further comprising:
oxidizing a second molybdenum electrode disposed in a second enclosed chamber of the phosphate level determination device, with a proton exchange member being disposed between the first enclosed chamber and the second enclosed chamber, to cause protons to migrate from the second enclosed chamber to the first enclosed chamber to reduce a pH level of the fluid disposed in the first enclosed chamber.
   35. The method of claim 34, wherein migration of the protons from the second enclosed chamber to the first enclosed chamber reduces the pH level of the fluid disposed in the first chamber to a range of about 0.8 to about 1.2.
   36. The method of claim 34 or claim 35, wherein the first molybdenum electrode and the working electrode are disposed directly adjacent to the second chamber.
   37. The method of any of claims 34 to 36, wherein a total chamber volume of the phosphate level detection device is about 6 microliters or less.
   38. The method of claim 32 or claim 33,
wherein the first molybdenum electrode is disposed in a first chamber of a phosphate level detection device, and
a total chamber volume of the phosphate level detection device is about 1.5 microliters or less.
   39. The method of any of claims 31 to 38, wherein approximately 0.08 milligrams of molybdenum or less is consumed per measurement.
   40. The method of any of claims 31 to 33 and 38, wherein approximately 0.0008 milligrams of molybdenum or less is consumed per measurement.
   41. The method of any of claims 31 to 40, wherein approximately 0.2 Joules or less of energy for each 1 millimeter² of exposed first molybdenum electrode surface area is consumed per measurement.
   42. The method of any of claims 31 to 33, 38, and 40, wherein 0.00025 Joules or less of energy for each 1 millimeter² of exposed first molybdenum electrode surface area is consumed per measurement.
   43. The method of any of claims 31 to 42, wherein determining a level of phosphate ions in the fluid from which the reagent is generated consumes approximately two minutes or less of time in the absence of stirring the fluid during the generating and determining actions.
   44. The method of any of claims 31 to 33, 38, 40, and 42, wherein determining a level of phosphate ions in the fluid from which the reagent is generated consumes approximately thirty seconds or less of time in the absence of stirring the fluid during the generating and determining actions.
   45. The method of claim 31, wherein determining a level of phosphate ions in the fluid from which the reagent is generated consumes approximately ten seconds or less of time in the absence of stirring the fluid during the generating and determining actions.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. A phosphate level detection device, comprising:
a first chamber;
a first molybdenum electrode at least partially disposed within the first chamber; and
a working electrode at least partially disposed within the first chamber, the working electrode being positioned within about 100 micrometers of the first molybdenum electrode,
wherein the device is configured such that oxidation of the first molybdenum electrode in the presence of a fluid that includes phosphate ions results in the formation of a 12-molybdophosphoric acid.

2. The phosphate level detection device of claim 1, further comprising:
a second chamber, each of the first chamber and the second chamber being enclosed chambers;
a second molybdenum electrode at least partially disposed in the second chamber; and
a proton exchange membrane disposed between the first chamber and the second chamber,
wherein the device is configured such that oxidation of the second molybdenum electrode in the presence of a fluid results in protons migrating from the second chamber, across the proton exchange membrane, and to the first chamber to reduce a pH level of fluid disposed in the first chamber.

3. The phosphate level detection device of claim 2, wherein the device is configured such that the protons that migrate from the second chamber, across the proton exchange membrane, and to the first chamber reduce the pH level to a range of about 0.8 to about 1.2.

4. The phosphate level detection device of claim 2, wherein the first molybdenum electrode and the working electrode are disposed directly adjacent to the proton exchange membrane.

5. The phosphate level detection device of claim 1, further comprising at least one of:
a reference electrode; or
a counter electrode,
wherein the at least one of the reference electrode or the counter electrode, in conjunction with the working electrode, are configured to make electrochemical determinations of a phosphate level of a fluid disposed in the first chamber when the first molybdenum electrode is oxidized.

6. The phosphate level detection device of claim 1, further comprising:
a reference electrode; and
a counter electrode;
wherein the first chamber is an open chamber and each of the first molybdenum electrode, the working electrode, the reference electrode, and the counter electrode are at least partially disposed within bounds defined by walls of the first chamber, the device being configured to operate in an open-cell configuration.

7. The phosphate level detection device of claim 1, wherein the device has a total chamber volume of about 6 microliters or less.

8. The phosphate level detection device of claim 1, wherein the device has a total chamber volume of about 1.5 microliters or less.

9. The phosphate level detection device of claim 1, wherein a molybdenum consumption level is approximately 0.08 milligrams or less per measurement.

10. The phosphate level detection device of claim 1, wherein a molybdenum consumption level is approximately 0.0008 milligrams or less per measurement.

11. The phosphate level detection device of claim 1, wherein an energy consumption level for oxidation of the first molybdenum electrode is approximately 0.2 Joules or less per measurement for each 1 millimeter² of exposed first molybdenum electrode surface area.

12. The phosphate level detection device of claim 1, wherein an energy consumption level for oxidation of the first molybdenum electrode is approximately 0.00025 Joules or less per measurement for each 1 millimeter² of exposed first molybdenum electrode surface area.

13. The phosphate level detection device of claim 1, wherein a phosphate level detection time of the device is approximately two minutes or less in the absence of stirring the 12-molybdophosphoric acid.

14. The phosphate level detection device of claim 1, wherein a phosphate level detection time of the device is approximately 30 seconds or less in the absence of stirring the 12-molydophosphoric acid.

15. The phosphate level detection device of claim 1, wherein a phosphate level detection time of the device is approximately 10 seconds or less in the absence of stirring the 12-molydophosphoric acid.

16. A method for determining phosphate levels in a fluid, comprising:
oxidizing a first molybdenum electrode that is at least partially disposed in a first chamber of a phosphate level detection device to form a 12-molybdophosphoric acid from a fluid having phosphate ions disposed therein; and
determining a level of phosphate ions present in the fluid from which the 12-molybdophosphoric acid is formed using an electrochemical set-up, the electrochemical set-up including a working electrode disposed within about 100 micrometers of the first molybdenum electrode.

17. The method of claim 16, further comprising:
causing the fluid having phosphate ions disposed therein to enter the first chamber,
wherein the fluid comes from a fluid source that is at a location at which the oxidizing and determining actions are performed such that determining a level of phosphate ions present in the fluid from which the 12-molybdophosphoric acid is formed occurs in situ.

18. The method of claim 16, further comprising:
oxidizing a second molybdenum electrode that is at least partially disposed in a second chamber to cause protons to migrate from the second chamber, to the first chamber, to reduce a pH level of the fluid disposed in the first chamber,
wherein each of the first and second chambers are enclosed.

19. The method of claim 18, further comprising:
causing the fluid having phosphate ions disposed therein to enter the second chamber,
wherein the fluid comes from a fluid source that is at a location at which the oxidizing and determining actions are performed such that determining a level of phosphate ions present in the fluid from which the 12-molybdophosphoric acid is formed occurs in situ.

20. The method of claim 18, wherein migration of the protons from the second chamber to the first chamber reduces the pH level of the fluid disposed in the first chamber to a range of about 0.8 to about 1.2.

21. The method of claim 18, wherein the first molybdenum electrode and the working electrode are disposed directly adjacent to the second chamber.

22. The method of claim 16, wherein a total chamber volume of the phosphate level detection device is about 6 microliters or less.

23. The method of claim 16, wherein a total chamber volume of the phosphate level detection device is about 1.5 microliters or less.

24. The method of claim 16, wherein approximately 0.08 milligrams of molybdenum or less is consumed per measurement.

25. The method of claim 16, wherein approximately 0.0008 milligrams of molybdenum or less is consumed per measurement.

26. The method of claim 16, wherein approximately 0.2 Joules or less of energy for each 1 millimeter² of exposed first molybdenum electrode surface area is consumed per measurement.

27. The method of claim 16, wherein approximately 0.00025 Joules or less of energy for each 1 millimeter² of exposed first molybdenum electrode surface area is consumed per measurement.

28. The method of claim 16, wherein determining a level of phosphate ions present in the fluid from which the 12-molybdophosphoric acid is formed using an electrochemical set-up consumes approximately two minutes or less of time in the absence of stirring the 12-molybdophosphoric acid.

29. The method of claim 16, wherein determining a level of phosphate ions present in the fluid from which the 12-molybdophosphoric acid is formed using an electrochemical set-up consumes approximately thirty seconds or less of time in the absence of stirring the 12-molybdophosphoric acid.

30. The method of claim 16, wherein determining a level of phosphate ions present in the fluid from which the 12-molybdophosphoric acid is formed using an electrochemical set-up consumes approximately ten seconds or less of time in the absence of stirring the 12-molybdophosphoric acid.

31. A method for determining phosphate levels in a fluid, comprising:
sampling a fluid having phosphate ions disposed therein from a fluid source;
generating a reagent from the fluid having phosphate ions disposed therein due to anodic dissolution of molybdenum metal; and
determining a level of phosphate ions in the fluid from which the reagent is generated,
wherein the actions of sampling, generating, and determining are all performed at the location of the fluid source such that determining a level of phosphate ions in the fluid from which the reagent is generated occurs in situ.

32. The method of claim 31, wherein generating a reagent from the fluid having phosphate ions disposed therein due to anodic dissolution of molybdenum metal further comprises:
oxidizing a first molybdenum electrode that includes the molybdenum metal that is dissolved.

33. The method of claim 32, wherein a working electrode used in conjunction with determining a level of phosphate ions in the fluid from which the reagent is generated is disposed within about 100 micrometers of the first molybdenum electrode.

34. The method of claim 32, wherein the first molybdenum electrode is disposed in a first enclosed chamber of a phosphate level determination device, the method further comprising:
oxidizing a second molybdenum electrode disposed in a second enclosed chamber of the phosphate level determination device, with a proton exchange member being disposed between the first enclosed chamber and the second enclosed chamber, to cause protons to migrate from the second enclosed chamber to the first enclosed chamber to reduce a pH level of the fluid disposed in the first enclosed chamber.

35. The method of claim 34, wherein migration of the protons from the second enclosed chamber to the first enclosed chamber reduces the pH level of the fluid disposed in the first chamber to a range of about 0.8 to about 1.2.

36. The method of claim 34, wherein the first molybdenum electrode and the working electrode are disposed directly adjacent to the second chamber.

37. The method of claim 34, wherein a total chamber volume of the phosphate level detection device is about 6 microliters or less.

38. The method of claim 32,
wherein the first molybdenum electrode is disposed in a first chamber of a phosphate level detection device, and
a total chamber volume of the phosphate level detection device is about 1.5 microliters or less.

39. The method of claim 31, wherein approximately 0.08 milligrams of molybdenum or less is consumed per measurement.

40. The method of claim 31, wherein approximately 0.0008 milligrams of molybdenum or less is consumed per measurement.

41. The method of claim 31, wherein approximately 0.2 Joules or less of energy for each 1 millimeter² of exposed first molybdenum electrode surface area is consumed per measurement.

42. The method of claim 31, wherein 0.00025 Joules or less of energy for each 1 millimeter² of exposed first molybdenum electrode surface area is consumed per measurement.

43. The method of claim 31, wherein determining a level of phosphate ions in the fluid from which the reagent is generated consumes approximately two minutes or less of time in the absence of stirring the fluid during the generating and determining actions.

44. The method of claim 31, wherein determining a level of phosphate ions in the fluid from which the reagent is generated consumes approximately thirty seconds or less of time in the absence of stirring the fluid during the generating and determining actions.

45. The method of claim 31, wherein determining a level of phosphate ions in the fluid from which the reagent is generated consumes approximately ten seconds or less of time in the absence of stirring the fluid during the generating and determining actions.
